(19) United States
(12) Patent Application Publication
(10) Pub. No.: US 2025/0288425 A1
(43) Pub. Date: Sep. 18, 2025

(54) TALAR IMPLANTS AND IMPLANT SYSTEMS

(71) Applicant: Paragon 28, Inc., Englewood, CO (US)

(72) Inventor: Luciano Bernardino BERTOLOTTI, Denver, CO (US)

(73) Assignee: Paragon 28, Inc., Englewood, CO (US)

(21) Appl. No.: 19/216,382

(22) Filed: May 22, 2025

Related U.S. Application Data
(63) Continuation of application No. PCT/US2023/081749, filed on Nov. 30, 2023.
(60) Provisional application No. 63/385,508, filed on Nov. 30, 2022.

(51) Int. Cl. A61F 2/42; A61B 17/064; A61B 17/56; A61B 17/86; A61F 2/30

(52) U.S. Cl. CPC A61F 2/4202; A61B 17/0642; A61B 17/562; A61B 17/86; A61F 2002/30113; A61F 2002/30326; A61F 2002/30331; A61F 2002/30604; A61F 2002/30622; A61F 2002/4207

(57) ABSTRACT

An implant system that includes an implant having a base portion, that has a body and at least a portion of which includes a lattice structure, and an upper portion configured to releasably couple with the base portion. The implant system includes at least one first fastener configured to be received and extend at least partially through at least one bore of the base portion, and at least one second fastener configured to be received within a cavity of the base portion. Also disclosed is an implant that includes a body that has various elements configured to facilitate arthrodesis of the subtalar joint and talonavicular joint. The body includes an upper surface that is configured to interface with the tibia of a patient, and a lower surface that is configured to interface with the subtalar joint and/or calcaneus of the patient.

### CROSS REFERENCE TO RELATED APPLICATION

This application is a bypass continuation of PCT Application No. PCT/US2023/081749, filed Nov. 30, 2023, and entitled “Talar Implants and Implant Systems,” which claims benefit of priority benefit under 35 U.S.C. § 119 (e) of U.S. Provisional Patent Application No. 63/385,508, filed on Nov. 30, 2022, and entitled “Talar Implants and Implant Systems,” the disclosures of each of these applications are hereby incorporated herein by reference in their entirety.

### FIELD

The present disclosure relates to implantable medical devices (e.g., implants) and implant systems for surgical procedures. The present disclosure relates to podiatric and orthopedic implants and implant systems related to the foot/ankle and/or procedures incorporating surrounding bones/soft tissue. More specifically, but not exclusively, the present disclosure relates to implants and implant systems for procedures of the ankle joint.

### BACKGROUND OF THE INVENTION

Many currently available implants, instruments, systems, and surgical methods for procedures involving the foot/ankle do not completely address the needs of patients. Additionally, many currently available implants, instruments, systems, and surgical methods for incorporation in procedures involving the foot/ankle, for example ankle arthroplasty procedures, fail to account for properties of joint anatomy and associated mechanical and kinematic movement patterns/capabilities.

### SUMMARY OF THE INVENTION

The present disclosure is directed toward implants and implant systems for procedures involving the foot and/or ankle. More specifically, the present disclosure is directed to implants and implant systems for ankle procedures.

One aspect of the present disclosure is directed to an implant system. The implant system includes an implant which includes a base portion, at least a portion of which includes a lattice structure, and an upper portion configured to releasably couple with the base portion. The implant system also includes at least one first fastener configured to be received within and extend at least partially therethrough at least one bore of the base portion, and at least one second fastener configured to be received at least partially within a cavity of the base portion.

### BRIEF DESCRIPTION OF THE DRAWINGS

The accompanying drawings, which are incorporated in and constitute a part of the specification, illustrate embodiments of the inventions and together with the detailed description herein, serve to explain the principles of the inventions. It is emphasized that, in accordance with the standard practice in the industry, various features may or may not be drawn to scale. In fact, the dimensions of the various features may be arbitrarily increased or reduced for clarity of discussion. The drawings are only for purposes of illustrating embodiments of inventions of the disclosure and are not to be construed as limiting the inventions.

FIG. 1 is an anterior, perspective view of an implant system implanted in an ankle of a patient, in accordance with the present disclosure;

FIG. 2 is a lateral view of the implant system of FIG. 1, implanted in an ankle of a patient, in accordance with the present disclosure;

FIG. 3 is a medial view of the implant system of FIG. 1, implanted in an ankle of a patient, in accordance with the present disclosure;

FIG. 4 is an anterior view of the implant system of FIG. 1, implanted in an ankle of a patient, in accordance with the present disclosure;

FIG. 5 is side view of the implant system of FIG. 1, in accordance with the present disclosure;

FIG. 6 is a side view of the implant of the implant system of FIG. 1, in accordance with the present disclosure;

FIG. 7 is a top view of the implant of FIG. 6 of the implant system of FIG. 1, in accordance with the present disclosure;

FIG. 8 is a front view of the implant of FIG. 6 of the implant system of FIG. 1, in accordance with the present disclosure;

FIG. 9 is a side, perspective, exploded view of the implant of FIG. 6 of the implant system of FIG. 1, in accordance with the present disclosure;

FIG. 10 is a top view of a portion of the implant of FIG. 6 of the implant system of FIG. 1, in accordance with the present disclosure;

FIG. 11 is a lateral perspective view of an alternative implant system implanted in an ankle of a patient, in accordance with the present disclosure;

FIG. 12 is a side view of the implant system of FIG. 11, in accordance with the present disclosure;

FIG. 13 a side view of an implant of the implant system of FIG. 11, in accordance with the present disclosure; and

FIG. 14 is a top view of the implant of FIG. 13 of the implant system of FIG. 11, in accordance with the present disclosure.

### DETAILED DESCRIPTION

In this detailed description and the following claims, the words proximal, distal, anterior, or plantar, posterior, or dorsal, medial, lateral, superior, and inferior are defined by their standard usage for indicating a particular part or portion of a bone or implant according to the relative disposition of the natural bone or directional terms of reference. For example, “proximal” means the portion of a device or implant nearest the torso, while “distal” indicates the portion of the device or implant farthest from the torso. As for directional terms, “anterior” is a direction towards the front side of the body, “posterior” means a direction towards the back side of the body, “medial” means towards the midline of the body, “lateral” is a direction towards the sides or away from the midline of the body, “superior” means a direction above and “inferior” means a direction below another object or structure. Further, specifically in regards to the foot, the term “dorsal” refers to the top of the foot and the term “plantar” refers the bottom of the foot.

Similarly, positions or directions may be used herein with reference to anatomical structures or surfaces. For example, as the current implants, devices, instrumentation, and methods are described herein with reference to use with the bones of the foot, the bones of the foot, ankle and lower leg may be used to describe the surfaces, positions, directions or orientations of the implants, devices, instrumentation, and methods. Further, the implants, devices, instrumentation, and methods, and the aspects, components, features and the like thereof, disclosed herein are described with respect to one side of the body for brevity purposes. However, as the human body is relatively symmetrical or mirrored about a line of symmetry (midline), it is hereby expressly contemplated that the implants, devices, instrumentation, and methods, and the aspects, components, features and the like thereof, described and/or illustrated herein may be changed, varied, modified, reconfigured or otherwise altered for use or association with another side of the body for a same or similar purpose without departing from the spirit and scope of the invention. For example, the implants, devices, instrumentation, and methods, and the aspects, components, features and the like thereof, described herein with respect to the right foot may be mirrored so that they likewise function with the left foot. Further, the implants, devices, instrumentation, and methods, and the aspects, components, features and the like thereof, disclosed herein are described with respect to the foot for brevity purposes, but it should be understood that the implants, devices, instrumentation, and methods may be used with other bones of the body having similar structures.

The instruments, implants, systems, assemblies, and related methods for maintaining, correcting, and/or resurfacing joint surfaces of the present disclosure may be similar to, such as include at least one feature or aspect of, the implants, systems, assemblies and related methods disclosed in disclosed U.S. Pat. No. 10, 117,749, issued on Nov. 6, 2018 and entitled Subtalar Joint Implant; European Patent No. 3756626 issued on Dec. 30, 2020 and entitled Subtalar Joint Implant, European Patent Application No. 15770960.1A filed on Jul. 15, 2020 and entitled Subtalar Joint Implant; U.S. Provisional Patent Application No. 63/155,100 filed on Mar. 1, 2021 and entitled Methods for Performing Arthroplasty of the Subtalar Joint; U.S. Provisional Patent Application No. 63/167,965 filed on Mar. 30, 2021 and entitled Orthopedic Implants and Methods; International PCT Application No. PCT/US2019/29009, filed on Apr. 24, 2019, and entitled Implants and Methods of Use and Assembly; International PCT Application No. PCT/US2019/64741, filed on Dec. 12, 2019, and entitled Implant System and Methods of Use; International PCT Application No. PCT/US2019/66336, filed on Dec. 13, 2019, and entitled Patient Specific Instrumentation and Methods of Use; and/or International PCT Application No. PCT/US2019/66408, filed on Dec. 13, 2019, and entitled Joint Replacement Alignment Guides, System, and Methods of Use and Assembly; and/or International PCT Application No. PCT/US2019/66149 filed on Dec. 13, 2019, and entitled Alignment Instruments and Methods for Use in Total Ankle Replacement; and/or International PCT Application No. PCT/US2019/66393, filed on Dec. 13, 2019, entitled Joint Replacement Alignment Guides, Systems, and Methods of Use and Assembly; and/or U.S. Provisional Patent Application No. 62/898,615, filed on Sep. 11, 2019, entitled Resection Guides, Sweeping Reamers, and Methods for Use in Total Ankle Replacement; and/or International PCT Application No. PCT/US2019/66398, files on Dec. 13, 2019, entitled Distractors Having Attachable Paddles, Impaction Devices, and Methods for Use in Total Ankle Replacement; and/or International PCT Application No. PCT/US2019/65025, filed on Dec. 6, 2019, entitled Trial Insert Assembly; and/or U.S. Provisional Patent Application No. 62/899,460 filed Sep. 12, 2019, entitled Total Ankle Replacement Surgical Method; and/or International PCT Application No. PCT/US2019/66404 filed on Dec. 13, 2019, entitled Instruments, Guides, and Related Methods for Total Ankle Replacement; all of which are hereby incorporated herein by reference in their entireties. Similarly, the instruments, implants, systems, assemblies, and related methods for maintaining, correcting, and/or resurfacing joint surfaces of the present disclosure may include one or more instrument (e.g., one or more insertion and/or implantation instruments) disclosed in International PCT Application No. PCT/US2019/29009, filed on Apr. 24, 2019, and entitled Implants and Methods of Use and Assembly; International PCT Application No. PCT/US2019/64741, filed on Dec. 12, 2019, and entitled Implant System and Methods of Use; International PCT Application No. PCT/US2019/66336, filed on Dec. 13, 2019, and entitled Patient Specific Instrumentation and Methods of Use; and/or International PCT Application No. PCT/US2019/66408, filed on Dec. 13, 2019, and entitled Joint Replacement Alignment Guides, System, and Methods of Use and Assembly; and/or International PCT Application No. PCT/US2019/66149 filed on Dec. 13, 2019, and entitled Alignment Instruments and Methods for Use in Total Ankle Replacement; and/or International PCT Application No. PCT/US2019/66393, filed on Dec. 13, 2019, entitled Joint Replacement Alignment Guides, Systems, and Methods of Use and Assembly; and/or U.S. Provisional Patent Application No. 62/898,615, filed on Sep. 11, 2019, entitled Resection Guides, Sweeping Reamers, and Methods for Use in Total Ankle Replacement; and/or International PCT Application No. PCT/US2019/66398, files on Dec. 13, 2019, entitled Distractors Having Attachable Paddles, Impaction Devices, and Methods for Use in Total Ankle Replacement; and/or International PCT Application No. PCT/US2019/65025, filed on Dec. 6, 2019, entitled Trial Insert Assembly; and/or U.S. Provisional Patent Application No. 62/899,460 filed Sep. 12, 2019, entitled Total Ankle Replacement Surgical Method; and/or International PCT Application No. PCT/US2019/66404 filed on Dec. 13, 2019, entitled Instruments, Guides, and Related Methods for Total Ankle Replacement; and/or U.S. patent application Ser. No. 16/672,505 filed Nov. 3, 2019 and titled Talus Formational And Implantation Method; and/or International PCT Application No. PCT/US2021/046920 and titled Whole Talus Implant and Method; and/or International PCT Application No. PCT/US2021/047117 and titled Implant for Focal Talus Defects and Method; all of which are hereby incorporated herein by reference in their entireties.

Referring to the drawings included herein, implants and implant systems are shown and described. It should be understood that one or more of the implants and/or components of implant systems shown and described herein may be implemented in conjunction with one or more of the other various implants or implant systems shown and described herein. Further, it should be understood that the implants and implant systems, shown herein—as well as components thereof—may be duplicated, eliminated, or otherwise combined/modified and incorporated in conjunction with the same or other systems including but not limited to those shown and described herein and those incorporated by reference previously herein.

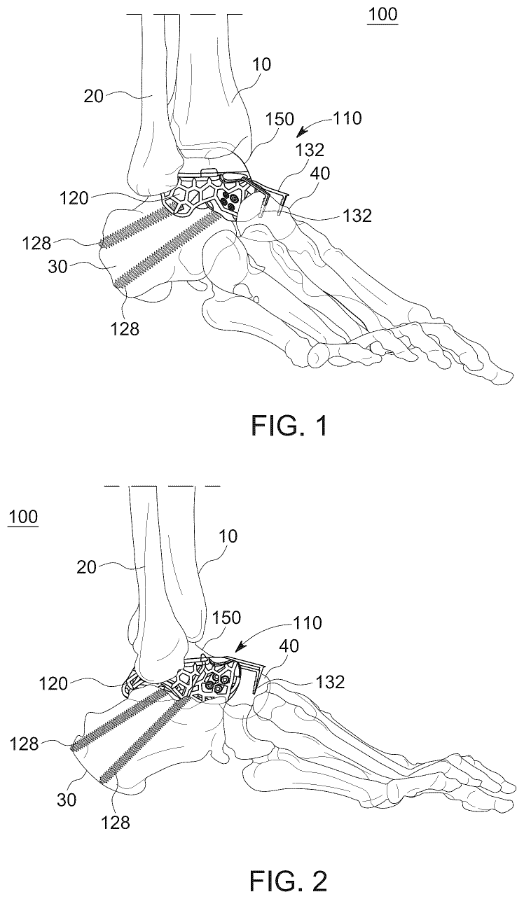

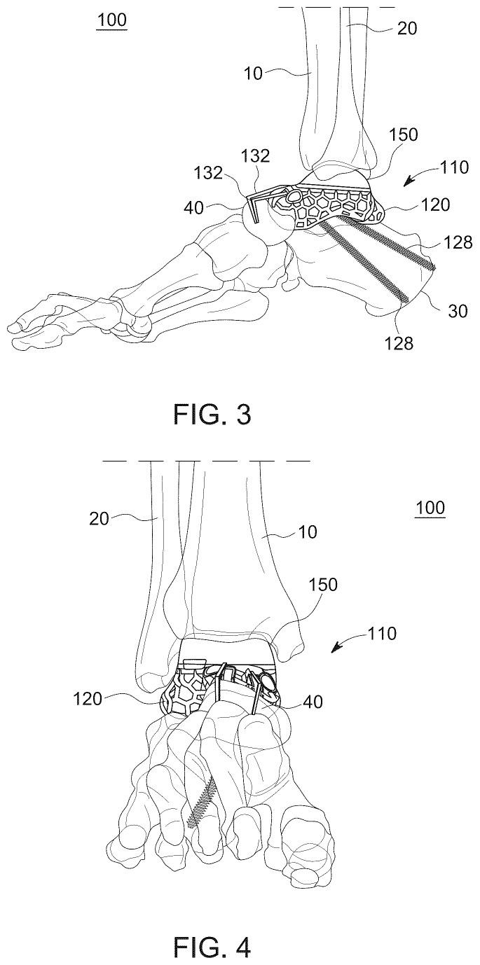

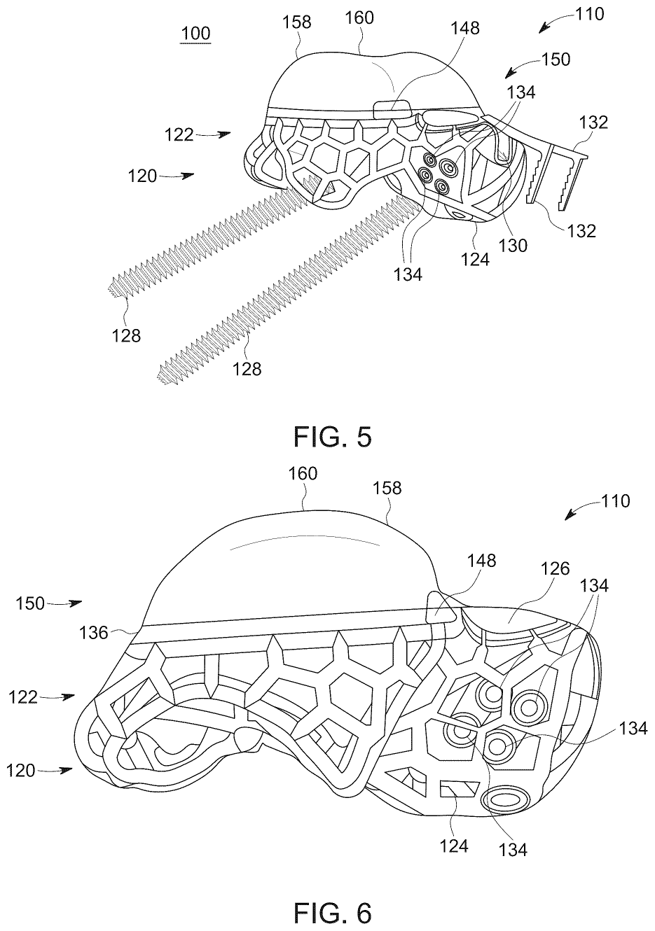

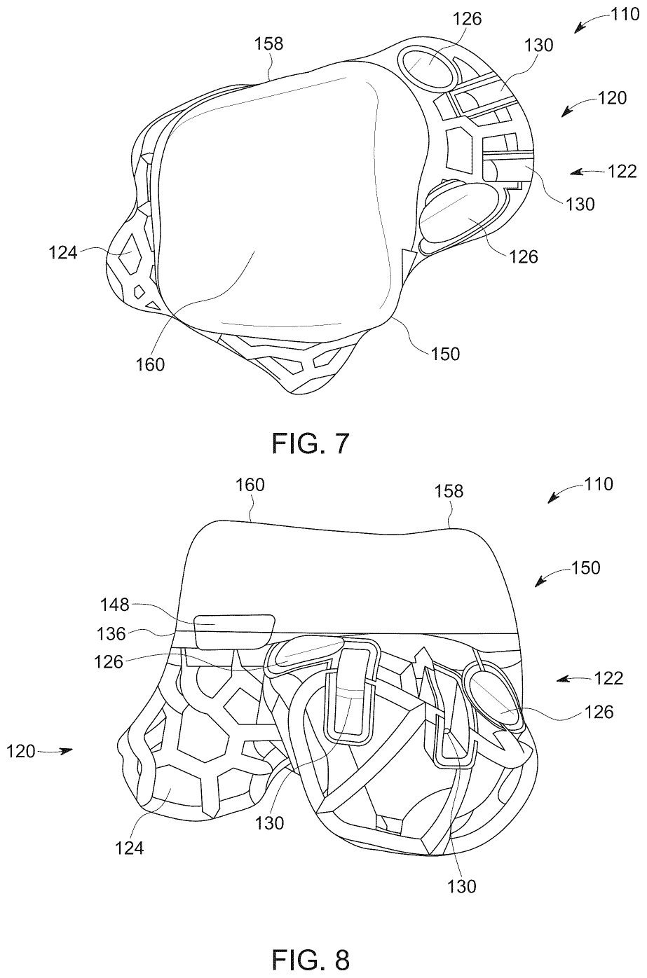

Referring now to FIGS. 1-10, an implant system 100 (referred to hereinafter as “system 100”) is shown adjacent the anatomy of the foot and ankle of a patient (e.g., a computer-generated model based on imaging data of anatomy of a patient, or a computer-generated model based on normalized values for anatomical structures). The anatomy shown includes (but is not limited to) structures of the ankle and hindfoot such as a tibia 10, a fibula 20, a calcaneus 30, and a navicular 40. As shown, the implant system 100 may include one or more components configured to interface with (e.g., releasably couple with, articulate against/with a surface thereof, be positioned adjacent, etc.) one or more of the aforementioned anatomical structures as shown and described in FIGS. 1-4. In some aspects, the system 100 may be implemented (e.g., prescribed and/or implanted by a physician) to treat various conditions and/or as a means for performing various procedures. For example, the system 100 may be implemented by a physician as a talar implant configured to replace a native talus of the patient. Further, the implant system 100 may also be implemented in order to facilitate arthrodesis of the subtalar and/or talonavicular joints in addition to replacing a native talus. The system 100 may also be manipulated and/or augmented (preoperatively, intraoperatively, and/or post-operatively in an additional procedure) so as to facilitate an arthroplasty procedure of the ankle and interface with one or more components of an ankle arthroplasty system. Similarly, the system 100 may be manipulated and/or augmented (preoperatively, intraoperatively, and/or post-operatively in an additional procedure) so as to facilitate an arthroplasty procedure of the ankle and interface with one or more components of an ankle fusion system. Accordingly, the system 100 may be implemented as shown in order to facilitate one or more arthrodesis procedures of the foot and/or ankle, or may be modified and/or implemented with additional orthopedic implants or systems (including but not limited to those incorporated by reference herein) to facilitate various procedures of the foot and/or ankle.

The system 100 is shown to include an implant 110 configured to replace a native talus of a patient. In some aspects, the implant 110 may be designed and subsequently produced via 3D printing or other production means (e.g., machining, additive manufacturing, etc.). In some aspects, the implant 110 may be designed according to one or more methods included in the pending patent applications incorporated by reference herein. For example, the implant 110 may have a size, shape, and one or more geometric features based on a volume identified in patient imaging (e.g., CT, MRI, etc.), where said volume is disposed between the tibia 10, fibula 20, calcaneus 30, and navicular 40 (e.g., in a space occupied by a native talus). In some aspects, the implant 110 may be available in various sizes (e.g., sizes 0-6, small-large, etc.) such that a physician may intraoperatively place one or more trials within a patient and, based on the fit of the trials, select an implant from the various sizes that best fits the patient.

The implant 110 is shown to include a base 120, with the base 120 including a body 122. The body 122 of the base 120 is shown to include a lattice structure 124, with the lattice structure 124 forming at least a portion of the base 120. In some aspects, the body 122 may include a frame structure that defines a shape of the implant 110, with the lattice structure 124 confined within the body 122 (e.g., such that the body portion 122 is proud relative to the lattice structure 124). Further, the lattice structure 124 may also be positioned within the body 122 such that at least a portion of the lattice structure 124 contacts one or more adjacent anatomical structures (e.g., the calcaneus 30, the navicular 40, etc.) upon implantation. In some aspects, the lattice structure 124 may be a randomized structure, or in some aspects may be a repeating structure. The lattice structure 124 as shown in FIGS. 1-10 extends out from and around a perimeter of a central portion of the body 122, although in some aspects the lattice structure 124 may occupy a majority and/or all of the body 122 of the base 120. In some aspects, the lattice structure 124 may be configured to facilitate bone ingrowth of the surrounding anatomy upon implantation, or may also be packed with one or more biologic substances.

The body 122 is further shown to include at least one bore 126 (shown and referenced herein as a pair of bores 126) extending through the body 122 at a substantially oblique angle relative to horizontal and vertical planes. As shown, the bores 126 have an upper-most portion at a front of the implant 110 and a lower-most portion at the rear of the implant 110 (e.g., sloping from anterior to posterior when the implant 110 is placed within the patient as shown in FIGS. 1-4). In some aspects, the bores 126 may be positioned on the implant 110 relative to the anatomy of the patient. For example, the bores 126 may be aligned along axes having a trajectory that, when the implant 110 is positioned within the patient, align with a portion of the calcaneus 30 of the patient.

Accordingly, as shown in FIGS. 1-8, the bores 126 are each shown to receive (and in some aspects, releasably couple with via internal threading or other releasable coupling mechanisms) at least partially within and/or therethrough a fastener 128 shown herein as a screw. In some aspects, the bores 126 may have parallel, converging, or diverging central axes which correspond to central axes of each of the fasteners 128 once placed. Further, in some aspects each of the bores 126 may be sized (in diameter, circumference, length, etc.) so as to accommodate a specific fastener 128 (e.g., diameter, length, etc.) based on the preference of a physician and/or the anatomy of the calcaneus 40 or surrounding bone structures of the patient. Upon implantation, the implant 110 may be placed in a position that is the same as and/or similar to that of the native talus of the patient such that the fasteners 128 may be placed into and extend through the bores 126 and be coupled with at least a portion of the calcaneus 30 so as to facilitate arthrodesis of the subtalar joint of the patient.

The body 122 is also shown to include at least one cavity 130 (shown and referenced herein as a pair of cavities 130) disposed on a front portion of the implant 110 (when implanted as shown in FIGS. 1-4) adjacent the bores 126. As shown, both of the cavities 130 are positioned laterally between the bores 126 and on a portion of the implant 110 that is positioned adjacent the navicular 40 (and, accordingly, the talonavicular joint) of the patient. Each of the cavities 130 are configured to receive at least a portion of a fastener 132 therein, shown herein as a staple. Each of the cavities 130 may include multiple geometries therein, for example a larger lateral dimension at an upper portion thereof and a lesser lateral dimension at a lower portion thereof. The larger lateral dimension may be configured to guide or ease positioning of at least a portion of the fastener 132 (e.g., a leg of the staple) (See FIGS. 1-3) into the upper portion of the cavity 130 such that the fastener 132 may then subsequently be manipulated so at least a portion of the fastener 132 (e.g., the leg) is positioned within the lower portion of the cavity 130. In some aspects, the lesser lateral dimension of the lower portion of the cavity 130 may be configured to facilitate coupling (releasable or otherwise) with the fastener 132, for example by a compression fit, interference fit, complimentary geometry of teeth or other engagement features of the fastener 132, or other engagement/coupling mechanisms. In some embodiments, the cavities 130 and fasteners 132 may be replaced with an additional bore or set of bores and corresponding fasteners the same as and/or similar to the bores 126 and fasteners 128, for example to facilitate arthrodesis of one or more joints of the medial column of the foot (e.g., with a beaming screw or other screw configurations).

The body 122 includes at least one bore 134 (shown in FIGS. 1-10 as a plurality of bores) positioned on a side of and extending into the body 122 of the implant 110. The bores 134 may include one or more engagement features thereon or therein, for example threading on one or more interior surfaces thereof. Each of the bores 134 may be configured to receive a soft tissue anchor (not shown) at least partially therein and, accordingly, may be configured (e.g., shape, diameter, depth, placement/position on the implant, angled, etc.) according to a preference of a physician (e.g., based on a size/shape of suture anchor the physician plans to implement) and/or relative to adjacent anatomy when the implant 110 is in an implanted position. Accordingly, a physician may couple at least a portion of a soft tissue anchor to a soft tissue structure of a patient and subsequently couple the soft tissue anchor with one of the bores 134 such that at least a portion of the soft tissue anchor is disposed at least partially within the bore 134 (or vice-versa). This process may be repeated multiple times for multiple soft tissue anchors, soft tissue structures, and/or bores 134 of the implant 110. In some aspects, the implant 110 may include an alternate number of bores 134 and/or alternate placement/position of the bores 134. For example, the bores 134 may be placed randomly about the body 122 of the implant 110 such that a physician may couple soft tissue anchors (and thus, soft tissue structures) with the implant 110.

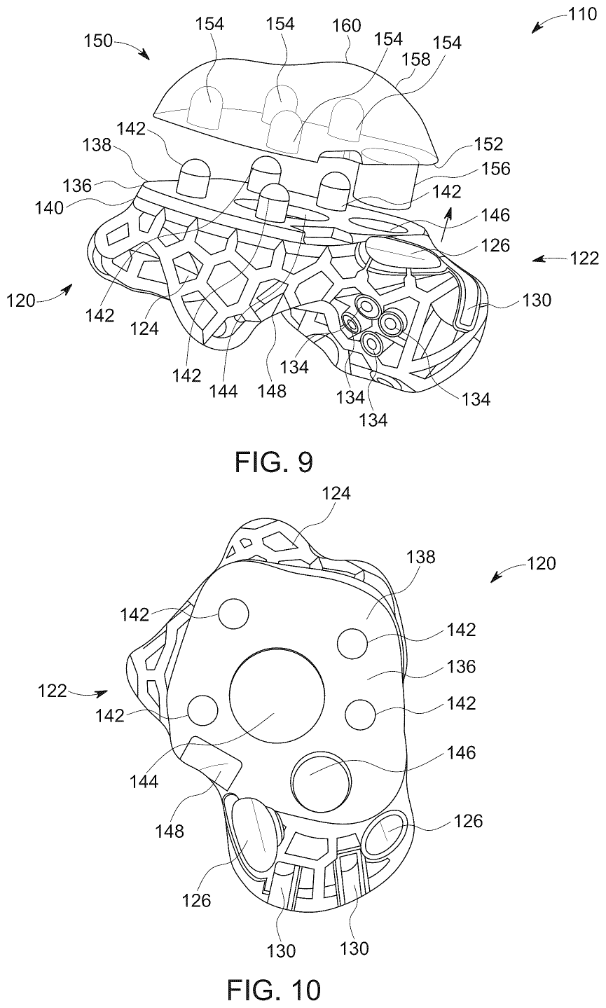

The base 120 of the implant 110 includes a plate 136 positioned above the body 122 of the implant 110. In some aspects, the plate 136 of the base 120 may be integral with the body 122, although in alternate embodiments, the plate 136 may be couplable with the body 122. As shown (See FIG. 9), a lower surface 140 of the plate 136 is shown to be integral with and/or contact the body 122, with an upper surface 138 positioned substantially opposite the plate 136 from the lower surface 140. The upper surface 138 of the plate 136 is shown to include at least one protrusion 142 (shown herein as a plurality of protrusions) disposed about a central opening 144. As shown, the protrusions 142 have a cylindrical/silo-shaped geometry and are spaced substantially equidistant from one another about the plate 136 and about (e.g., surrounding) the central opening 144. In some aspects, the quantity, spacing, size, geometry, or other properties of the protrusions 142 may be altered according to a specific procedure, physician preference, or anatomy of the patient. For example, in some aspects, the protrusions 142 on a first side of the plate 136 may be taller, shorter, or differently spaced/placed than the protrusions 142 on a second side of the plate 136 so as to adjust a deformity or imbalance of the patient (e.g., varus/valgus deformities, etc.). Further, in some aspects the protrusions 142 may be modified to facilitate compatibility (e.g., by providing a complimentary geometry) with various components of various implant systems (e.g., upper portions of the implant 110, talar components of ankle arthroplasty systems, etc.).

As shown in FIG. 10, the central opening 144 extends through the plate 136 from the top surface 138 to the bottom surface 140. Further, the central opening 144 also extends vertically through the body 122 of the base 120 of the implant and, as shown, includes a substantially circular/cylindrical geometry at its smallest lateral dimension (e.g., the central opening may have a greater lateral dimension as it extends through the body 122 than as it extends through the plate 136). In some aspects, the central opening 144 may be configured to accommodate at least a portion of an intramedullary (e.g., “IM”) nail therethrough. For example, if the system 100 and implant 110 were implanted as shown in FIGS. 1-4 and a physician preferred to convert the implant 110 and system 100 to an ankle (and/or hindfoot/tibiotalocalcaneal) fusion system, an IM nail may be placed such that it extends from anatomy inferior to the implant 110 (e.g., the calcaneus 30) through the central opening 144 and into anatomy superior to the implant 110 (e.g., the tibia 10). The base 120 of the implant 110 may be augmented and/or supplemented by positioning and/or coupling an additional implant component superior relative to the plate 136 such that the additional implant component is couplable with the plate 136 of the base 120.

The plate 136 is also shown to include a depression 146 disposed on the upper surface 138 thereof (See FIG. 10) and extending downward toward the body 122 of the base 122. As shown, the depression 146 is positioned adjacent the protrusions 142 at a front portion of the implant 110 when in an implanted position such as that shown in FIGS. 1-4. However, in some aspects, the depression 146 may be positioned variously about the upper surface 138 of the plate 136. In some aspects, the plate 136 may include multiple depressions 146 positioned variously about the plate 136, where one or more of said depressions 146 may vary in size, shape, and geometry from the depression 146 as shown in FIGS. 9-10. The depression 146 may be configured to receive a component of complimentary geometry at least partially therein so as to prevent rotation of a component including the complimentary geometry from rotating relative to the base 122, or vice-versa. The depression 146 may also include one or more engagement features therein, for example threading, ball detent-related geometries, etc.). In some aspects, the plate 136 may include alternate geometries to that shown with respect to the protrusions 142 and the depression 146. For example, the plate 136 may include a plurality of depressions 146 sized and positioned similarly to the protrusions 142 as shown, as well as one or more protrusions sized and positioned similarly to the depression 146.

The plate 136 also includes a notch 148 positioned adjacent to the depression 146 (e.g., near a front/anterior portion of the implant 110 when in the implanted position of FIGS. 1-4). In some aspects, the notch 148 (See FIG. 10) may include a change in elevation along a set distance (e.g., a drop, etc.) in the upper surface 138 of the plate 136. In some aspects, one or more dimensions of the notch 148 may correspond to an instrument (either common to orthopedic procedures such as an osteotome, or provided with the system 100 and having a geometry complimentary to the notch 148) configured to be positioned at least partially within and/or adjacent to the notch as to manipulate (e.g., separate, decouple, etc.) another component of the system 100 coupled with the base 120. In some aspects, the notch 148 may align with a corresponding, complimentary notch 148 in a component of the system 100 that is positioned superior to the plate 136. Further to the previous example, such a complimentary notch in said component may include a change in elevation (e.g., an increase, etc.) in the lower surface of said component.

The system 100 and the implant 110 include a modular portion 150 (e.g., upper portion) (See FIG. 9) configured to releasably couple with the base 120. The modular portion is shown to include a lower surface 152 configured substantially opposite the modular portion 150 from an upper surface 158. The upper surface 158 is shown to include an articulation surface 160 and, in some aspects, the articulation surface 160 may be configured to interface with at least a portion of the native anatomy of the ankle joint (and/or adjacent anatomical structures) including, for example, the tibia 10 and associated soft tissue structures. Accordingly, the articulation surface 160 may be specific to the anatomy of the patient and have a geometry complimentary to native structures. Further, the articulation surface 160 may also be configured to interface with (e.g., have a complimentary geometry or geometry configured to facilitate contact or adjacent movement with/between) one or more components of an implant or implant system, for example one or more components of an ankle arthroplasty system including but not limited to those incorporated by reference herein. Accordingly, the articulation surface 160 may be configured specifically to articulate (e.g., contact, be positioned adjacent, etc.) with a surface of any of the aforementioned implants or implant systems.

Referring now to FIG. 9, the modular portion 150 is shown to include at least one depression 154 (shown and referenced herein as “depressions 154”) disposed on and extending below the lower surface 152 thereof. As shown, each of the depressions 154 correspond in position and geometry (e.g., have a substantially complimentary geometry) to the protrusions 142 disposed on the plate 136. In some aspects, one or more of the depressions 154 may include one or more engagement features (e.g., threading, etc.) therein complimentary to any engagement features of the protrusions 142 and configured to facilitate coupling with the base 120 of the implant 110 via the protrusions 142. Similarly, the lower surface 152 also includes a protrusion 156 extending therefrom and having a position and geometry (e.g., a substantially complimentary geometry) to the depression 146 of the plate 136. In some aspects, the modular portion 150 may include multiple protrusions 156 extending from the lower surface 152. Further, in some aspects the protrusion 156 may include one or more engagement features thereon configured to interface with complimentary engagement features of the depression 146.

When the implant 110 is in a coupled configuration, for example as shown in FIGS. 1-8, each of the protrusions 142 are at least partially received and positioned within each of the depressions 154. Similarly, in such a coupled configuration, the protrusion 156 is at least partially received and positioned within the depression 146. Collectively, the protrusions 142, 156 and the depressions 146, 154 enable releasable coupling between the modular portion 150 and the base 120. Further, engagement between the protrusions 142, 156 and the depressions 146, 154 restrict movement (e.g., rotation, translation, etc.) of the modular portion 150 relative to the base 120, and vice versa. As shown in FIG. 8, the notch 148 is positioned adjacent to the interfacing surfaces of the implant 110 in the coupled configuration and, accordingly, may receive at least part of an instrument therein to facilitate decoupling of the implant 110.

As mentioned previously, the implant system 100 is configured to be converted so as to facilitate alternative procedures without removing the base 120 from the patient. For example, if a physician elected to convert the implant 110 into a component of an ankle arthroplasty system, the physician may decouple the modular portion 150 from the base 120 by inserting and manipulating an instrument at least partially within the notch 148. The physician may then subsequently remove and replace the modular portion 150 with a modified modular portion having one or more features that are the same as and/or similar to that of the modular portion 150 (e.g., depressions 154 and protrusion 156) such that the modified modular portion is configured to couple with the base 120, but includes an articulation surface (different from the articulation surface 160) configured to interface with a specific sized component of a specific ankle arthroplasty system (e.g., a poly insert that couples with a tibial portion of an ankle arthroplasty system). In another example, a physician may elect to convert the implant system 100 into an arthrodesis system, for example, fusing the ankle and subtalar joints (or re-fusing the subtalar joint). The physician may decouple the modular portion 150 from the base 120 by inserting and manipulating an instrument at least partially within the notch 148. The physician may then remove and replace the modular portion 150 with an alternate modular portion, for example a modular portion configured to couple with the base 120. The alternate modular portion may also include a lattice structure (the same as and/or similar to the lattice structure 124) and a central opening having a central axis that, when coupled with the base 120, aligns with a central axis of the central opening 144. Accordingly, the physician may then place an IM nail through the desired anatomy (e.g., calcaneus 30, tibia 10, etc.) such that the IM nail is at least partially received in and extending through the central opening 144 and the coaxial opening of the alternate modular portion.

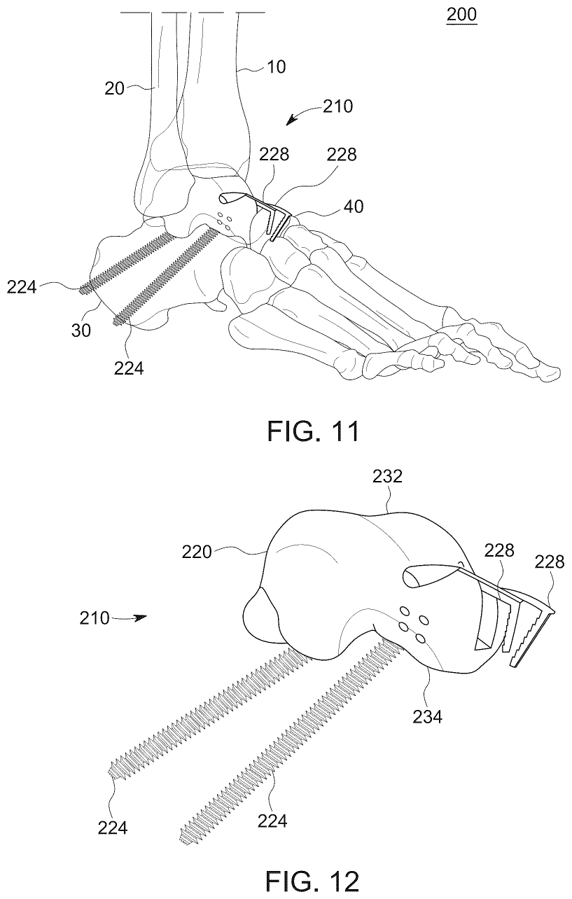

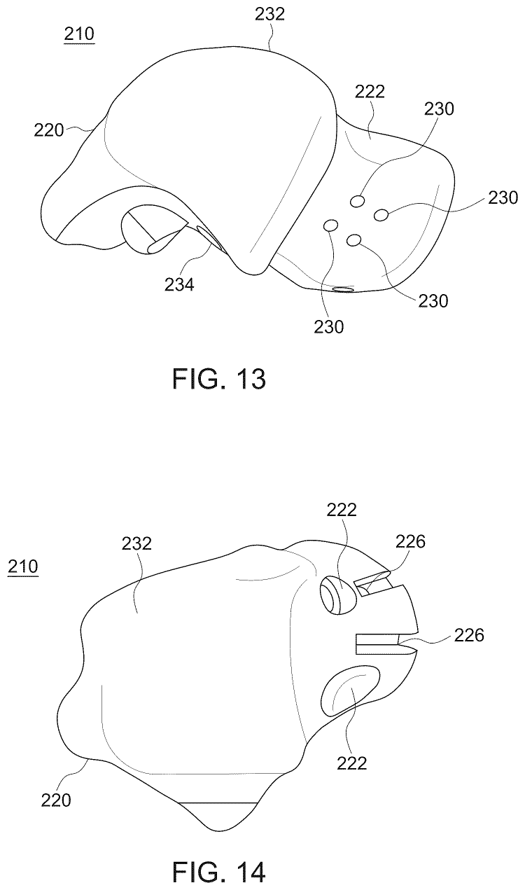

Referring now to FIGS. 11-14, an implant system 200 (referred to hereinafter as “system 200”) is shown, according to an exemplary embodiment. The system 200 is shown in FIG. 11 in an implanted configuration that is the same as or similar to the implanted configuration of the system 100 shown in FIGS. 1-4. The implant system 200 is shown to include an implant 210 which may have the same or similar geometry as the implant 110 of the system 100 and is configured to replace a native talus of a patient. Similarly, the implant 210 may be implanted in the same or similar fashion to the implant 110 and, accordingly, may interface with the same and/or similar anatomic structures. The implant 210 includes a body 220, with the body 220 including various features/components/geometries configured to facilitate arthrodesis of the subtalar joint, talonavicular joint, and support adjacent soft tissue structures. The body includes an upper surface 232 and a lower surface 234 and, as shown, the lower surface 234 is configured to interface with the subtalar joint and/or calcaneus 30 of a patient. The upper surface 232 is configured to interface with the tibia 10 and/or ankle joint of a patient. However, in some aspects the upper surface 232 may be configured to interface with one or more components of an ankle arthroplasty system and, accordingly, may have a geometry complimentary to said component(s) so as to facilitate articulation.

The body 220 is further shown to include at least one bore 222 (shown and referenced herein as a pair of bores 222) extending through the body 220 at a substantially oblique angle relative to horizontal and vertical planes. As shown, the bores 222 have an upper-most portion at the front of the implant 210 and a lower-most portion at the rear of the implant 210 (e.g., sloping from anterior to posterior when the implant 210 is placed within the patient as shown in FIG. 11). In some aspects, the bores 222 may be positioned on the implant 210 relative to the anatomy of the patient. For example, the bores 222 may be aligned along axes having a trajectory that, when the implant 210 is positioned within the patient, align with a portion of the calcaneus 30 of the patient. Accordingly, as shown in FIGS. 11-12, the bores 222 are each shown to receive (and in some aspects, releasably couple with via internal threading or other releasable coupling mechanisms) at least partially within and/or therethrough a fastener 224 shown herein as a screw. In some aspects, the bores 222 may have parallel, converging, or diverging central axes which correspond to central axes of each of the fasteners 224 once placed. Further, in some aspects each of the bores 222 may be sized (in diameter, circumference, length, etc.) so as to accommodate a specific fastener 224 (e.g., diameter, length, etc.) based on the preference of a physician and/or the anatomy of the calcaneus 40 or surrounding structures of the patient. Upon implantation, the implant 210 may be placed in a position the same as and/or similar to that of the native talus of the patient such that the fasteners 224 may be placed into and extend through the bores 222 and be coupled with at least a portion of the calcaneus 30 so as to facilitate arthrodesis of the subtalar joint of the patient.

The body 220 is also shown to include at least one cavity 226 (shown and referenced herein as a pair of cavities 226) disposed on a front portion of the implant 210 (when implanted as shown in FIG. 11) adjacent the bores 222. As shown, both of the cavities 226 are positioned laterally between the bores 222 and on a portion of the implant 210 that is positioned adjacent the navicular 40 (and, accordingly, the talonavicular joint) of the patient. Each of the cavities 226 is configured to receive at least a portion of a fastener 228 therein, shown herein for example as a staple. Each of the cavities 226 may include multiple geometries therein, for example, a larger lateral dimension at an upper portion thereof and a lesser lateral dimension at a lower portion thereof. The larger lateral dimension may be configured to guide or ease positioning of at least a portion of the fastener 228 (e.g., a leg of the staple) into the upper portion of the cavity 226 such that the fastener 228 may then subsequently be manipulated such that at least a portion of the fastener 228 (e.g., the leg) is positioned within the lower portion of the cavity 226. In some aspects, the lesser lateral dimension of the lower portion of the cavity 226 may be configured to facilitate coupling (releasable or otherwise) with the fastener 228, for example by a compression fit, interference fit, complimentary geometry of the teeth or other engagement features of the fastener 228, or other engagement/coupling mechanisms.

The body 220 includes at least one bore 230 (shown in FIGS. 11-14 as a plurality of bores) positioned on a side of and extending into the body 220 of the implant 210. The bores 230 may include one or more engagement features thereon or therein, for example threading on one or more interior surfaces thereof. Each of the bores 230 may be configured to receive a soft tissue anchor (not shown) at least partially therein and, accordingly, may be configured (e.g., shape, diameter, depth, placement/position on the implant, angled, etc.) according to a preference of a physician (e.g., based on a size/shape of suture anchor the physician plans to implement) and/or relative to the adjacent anatomy when the implant 210 is in an implanted position. Accordingly, a physician may couple at least a portion of a soft tissue anchor to a soft tissue structure of a patient and subsequently couple the soft tissue anchor with one of the bores 230 such that at least a portion of the soft tissue anchor is disposed at least partially within the bore 230 (or vice-versa). This process may be repeated multiple times for multiple soft tissue anchors, soft tissue structures, and/or bores 230 of the implant 210. In some aspects, the implant 210 may include an alternate number of bores 230 and/or alternate placement of the bores 230. For example, the bores 230 may be placed variously about the body 220 of the implant 210 such that a physician may couple soft tissue anchors (and thus, soft tissue structures) with the implant 210.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprise” (and any form of comprise, such as “comprises” and “comprising”), “have” (and any form of have, such as “has”, and “having”), “include” (and any form of include, such as “includes” and “including”), and “contain” (and any form of contain, such as “contains” and “containing”) are open-ended linking verbs. As a result, a method or device that “comprises,” “has,” “includes,” or “contains” one or more steps or elements possesses those one or more steps or elements, but is not limited to possessing only those one or more steps or elements. Likewise, a step of a method or an element of a device that “comprises,” “has,” “includes,” or “contains” one or more features possesses those one or more features, but is not limited to possessing only those one or more features. Furthermore, a device or structure that is configured in a certain way is configured in at least that way, but may also be configured in ways that are not listed.

The invention has been described with reference to the preferred embodiments. It will be understood that the architectural and operational embodiments described herein are exemplary of a plurality of possible arrangements to provide the same general features, characteristics, and general system operation. Modifications and alterations will occur to others upon a reading and understanding of the preceding detailed description. It is intended that the invention be construed as including all such modifications and alterations.

What is claimed is:

1. An implant system, comprising:
an implant, comprising:
a base portion, wherein at least a portion of the base portion comprises a lattice structure and a body; and
an upper portion configured to releasably couple with the base portion.

2. The implant system of claim 1, wherein the implant system further comprises
at least one first fastener configured to be received within and extend at least partially therethrough at least one bore of the body; and
at least one second fastener configured to be received at least partially within a cavity of the base portion.

3. The implant system of claim 2, wherein the at least one first fastener and the at least second fastener are a plurality of fasteners.

4. The implant system of claim 2, wherein the at least one bore is a pair of bores.

5. The implant system of claim 4, wherein the pair of bores extend through the body at an oblique angle relative to at least one of a horizontal plane or a vertical plane.

6. The implant system of claim 4, wherein the pair of bores comprise an upper portion and a lower portion, wherein the upper portion is positioned at an anterior aspect of the implant and the lower portion is positioned at a posterior aspect of the implant.

7. The implant system of claim 4, wherein the pair of bores slope in an anterior to posterior direction to facilitate arthrodesis of a subtalar joint.

8. The implant system of claim 1, wherein the body further comprises at least one cavity, wherein the at least one cavity is positioned at an anterior aspect of the implant and is sized to receive a staple fastener.

9. The implant system of claim 9, wherein the staple fastener is positioned and sized to facilitate arthrodesis of a talonavicular joint.

10. The implant system of claim 1, wherein the base portion further comprises a plate member, wherein the plate member comprises an upper surface and a lower surface, wherein the lower surface is at least one of couplable or integral to the body.

11. The implant system of claim 10, wherein the upper surface of the plate member comprises at least one protrusion.

12. The implant system of claim 11, wherein the at least one protrusion is a plurality of protrusions, wherein the plurality of protrusions each have a cylindrical or silo shaped geometry.

13. The implant system of claim 1, wherein the lattice structure of the base portion comprises a plurality of struts and openings positioned adjacent to each other in a web-like arrangement, wherein the struts and openings are sized to facilitate bone ingrowth.

14. The implant system of claim 10, wherein the upper surface of the plate member comprises a notch, wherein the notch is positioned proximate to the anterior aspect of the implant.

15. The implant system of claim 14, wherein the notch is sized to receive a tip of an instrument to facilitate the separation of the base portion from the upper portion.

16. The implant system of claim 1, wherein the upper portion comprises a lower surface and an upper surface, wherein the lower surface is configured to couple to the upper surface of the plate member.

17. The implant system of claim 16, wherein the lower surface of the upper portion includes a plurality of depressions, wherein the plurality of depressions are configured to align and receive the plurality of the protrusions of the upper surface of the plate member to releasably couple the base portion to the upper portion.

18. The implant system of claim 16, wherein the upper surface of the upper portion is substantially convex to facilitate articulation with a corresponding tibial articulating surface.

19. The implant system of claim 1, wherein the upper portion is modular relative to the base portion.

20. The implant system of claim 1, wherein the upper portion is a plurality of upper portions, wherein the plurality of upper portions comprise different geometries of upper surfaces, different heights and different outer shape configurations.

21. A modular implant comprising:
a base portion, wherein at least a portion of the base portion comprises a lattice structure and a body, wherein the body includes a plate member;
an upper portion configured to releasably couple to the plate member, wherein the upper portion includes an upper surface configured to articulate with an anatomic surface of a patient;
at least one fastener configured to be received within and extend at least partially through at least one bore of the base portion; and
at least one fastener configured to be received at least partially within a cavity of the base portion.
